(19) United States
(12) Patent Application Publication
(10) Pub. No.: US 2019/0170756 A1
(43) Pub. Date: Jun. 6, 2019

(54) TIRC7 BASED DIAGNOSTIC AND THERAPY OF SOLID CANCER

(71) Applicant: Nekonal S.a.r.l., Luxembourg (LU)

(72) Inventor: Nalan UTKU, Munich (DE)

(21) Appl. No.: 16/324,393

(22) PCT Filed: Aug. 10, 2017
(86) PCT No.: PCT/EP2017/070311

§ 371 Date: Feb. 8, 2019

(30) Foreign Application Priority Data
Aug. 10, 2016 (EP) 16183578.0

(51) Int. Cl. G01N 33/574; C07K 16/30; A61P 35/00

(52) U.S. Cl. CPC G01N 33/57492; C07K 16/3061; G01N 33/57496; A61P 35/00; A61K 45/06; C12Q 2600/112; C12Q 2600/158; G01N 2800/52; C12Q 2600/106

(57) ABSTRACT

The present invention pertains to a combined diagnostic and therapeutic approach for cancer patients comprising the analysis of the biomarker T cell immune response cDNA 7 (TIRC7) and, depending on its expression in tumor cells, tumor infiltrating lymphocytes (TIL) and T regulatory cells (Treg) in a patient, the use of TIRC7 as a target for therapy. In particular, the invention provides methods for stratifying patients with cancer into two groups one of which will benefit from a TIRC7 modulatory treatment and one of which are non-responders to such a therapy. Furthermore, the invention provides compounds for the treatment of patients that are identified as responders according to the invention. Thus, the present disclosure offers a true theranostic approach for patients with various cancers via tackling immune escape of the cancer, in particular solid tumors, based on TIRC7.

### FIELD OF THE INVENTION

The present invention pertains to a combined diagnostic and therapeutic approach for cancer patients comprising the analysis of the biomarker T cell immune response cDNA 7 (TIRC7) and, depending on its expression in tumor cells, tumor infiltrating lymphocytes (TIL) and T regulatory cells (Treg) in a patient, the use of TIRC7 as a target for therapy. In particular, the invention provides methods for stratifying patients with cancer into two groups one of which will benefit from a TIRC7 modulatory treatment and one of which are non-responders to such a therapy. Furthermore, the invention provides compounds for the treatment of patients that are identified as responders according to the invention. Thus, the present disclosure offers a true theranostic approach for patients with various cancers via tackling immune escape of the cancer, in particular solid tumors, based on TIRC7.

### DESCRIPTION

Estimates of the worldwide incidence, mortality and prevalence of cancer in the year 2002, show 10,9 million new cases, 7,6 million deaths, and 24,6 million persons alive with cancer [Parkin D M 2005]. Currently available treatments have limitations and treatment success is limited to a subgroup of patients with certain cancers leaving a large group of patients with high therapeutic unmet need. Thus, novel drugs and treatment strategies are major objectives of research; conjugates of antibodies with powerful cytotoxic agents have been explored.

Circulating T cells that specifically target normal self-proteins expressed by regulatory immune cells were first described in patients with cancer, but can also be detected in healthy individuals. The adaptive immune system is distinguished for its ability to differentiate between self-antigens and foreign antigens. Thus, it was remarkable to discover T cells that apparently lacked tolerance to important self-proteins, eg, IDO, PD-L1, and FoxP3, expressed in regulatory immune cells. The ability of self-reactive T cells to react to and eliminate regulatory immune cells can influence general immune reactions termed antiregulatory T cells (antiTregs).

Utilizing anti-Tregs for anticancer immunotherapy implies the direct targeting of cancer cells in addition to regulatory immune cells. Anti-Tregs provide the immune system with yet another level of immune regulation and contradict the notion that immune cells involved in the adjustment of immune responses only act as suppressor cells. Many regulatory mechanisms control the termination of immune responses to ensure unresponsiveness or tolerance to selfantigens. However, the immune regulation mechanisms that prevent autoimmunity may be harnessed by cancer cells to accomplish immune escape. This phenomenon was highlighted in the recently updated version of The Hallmarks of Cancer by Hanahan and Weinberg; now, “evasion of immune destruction” is listed as an emerging hallmark of cancer. Cancer cells can directly suppress anticancer immune mechanisms. In addition, cancer cells attract and/or convert immune-competent cells to generate and uphold an immunepermissive microenvironment. For example, tumor cells can escape from immune surveillance by usurping local regulatory T cells, dendritic cell subtypes, myeloid-derived suppressor cells, and M2 or tumor-associated macrophages. Under normal physiological conditions, these immune cells are involved in maintaining immune homeostasis, but in cancerous conditions, they become involved in the creation of an immunosuppressive microenvironment around tumors. Impressive clinical responses have been achieved by characterizing inhibitory T cell pathways and targeting them with monoclonal antibodies against specific membrane proteins (eg, CTLA-4, PD-1, or PD-L1) self-reactive T cells that specifically recognized human leukocyte antigen (HLA)-restricted epitopes derived from proteins expressed in regulatory immune cells, eg, indoleamine 2,3-dioxygenase (IDO), IDO2, tryptophan 2,3-dioxygenase (TDO), programmed death ligand 1 (PD-L1), heme-oxygenase-1 (HO-1) and forkhead box P3 (Foxp3). PD-L1 expression can be induced by IFNs, which are found in inflammatory microenvironments. Hence, PD-1 and its ligands play a central role in maintaining peripheral tolerance and preventing autoimmunity. In multiple cancers high PD-L1 expression has been described both on malignant cells as well as other cells in the tumor microenvironment. Thus, cancer cells exploit this system to create an immune suppressive microenvironment, which is protecting them from immune surveillance.

PD-L1 expression was first described as an indicator of tumor aggressiveness in renal cell carcinoma. In addition, PD-L1 expression on tumor cells has been suggested as a prognostic factor in a number of solid cancers including ovarian cancer and pancreatic cancer. Additionally, surface expression of PD-L1 on cancer cells has been described in several hematological cancers. Natural PD-L1-reactive anti-Tregs were readily isolated from the peripheral blood of patients with cancer. Thus, PD-L1-specific anti-Tregs are another example of the ability of the immune system to react directly against immune-suppressive mechanisms that have been adopted by cancerous cells.

A potential candidate for the development of novel therapeutic strategies is the cell surface protein, TIRC7 (T cell immune response cDNA7) induced after immune activation in subset of human T-, B-cells as well as monocytes and dendritic cells and Tregs. During immune activation, TIRC7 is co-localized with the T cell receptor and CTLA4 within the immune synapse of human T cells. At protein and mRNA level, its expression is induced in lymphocytes in synovial tissues obtained from patients with rheumatoid arthritis or during rejection of solid organ transplants and bone marrow transplantation as well as in brain tissues obtained from patients with multiple sclerosis.

In view of the above drawback in the diagnostic and treatment of malignancies, it was an object of the present invention to provide novel combined diagnostic and therapeutic approaches for patients suffering from solid tumors. The invention seeks to provide novel options for patients with cancer diseases, in particular such patients that already received therapy with anti-PD1, anti-CD20, anti-CTLA4 and/or anti-PDL1 and have become refractory, and/or have become resistant to available therapies on the market. In particular the present invention seeks to overcome immune escape mechanisms of cancers via targeting the immune cells harnessed by the tumor.

Thus, the above problem is first solved by a method for stratifying a cancer patient into one of patient groups (i) or (ii), wherein patient group (i) is a cancer patient in a group that will benefit from a T cell immune response cDNA 7 (TIRC7)-modulatory treatment, and patient group (ii) is a cancer patient group that will not benefit from a TIRC7 modulatory treatment, the method comprising the method steps of
(a) Providing a sample comprising a tumor tissue from said tumor in the cancer patient,
(b) Determining the expression of TIRC7 in a tumor cell in the tumor tissue or in an immune cell, such as a TIL, in the tumor tissue,
(c) Depending on the resultant of step (b), stratifying the patient into group (i) in the event the tumor cell in the tumor tissue and/or the immune cell in the tumor tissue expresses TIRC7 compared to a control cell, or stratifying the patient into group (ii) in the event the tumor cell in the tumor tissue and/or the immune cell in the tumor tissue does not express TIRC7 compared to a control cell.

In an alternative aspect the invention provides a method for diagnosing a cancer patient to have a solid cancer disease which will respond to a TIRC7 modulatory treatment, the method comprising the method steps of
(a) Providing a sample comprising a tumor tissue from said tumor in the cancer patient,
(b) Determining the expression of TIRC7 in a tumor cell in the tumor tissue or in an immune cell, such as a TIL, in the tumor tissue,
(c) Depending on the resultant of step (b), diagnosing the patient to have a solid cancer disease which will respond to a TIRC7 modulatory treatment in the event the tumor cell in the tumor tissue and/or the immune cell in the tumor tissue expresses TIRC7 compared to a control cell.

Preferred embodiments of the methods of the invention require that at least the immune cell in the tumor tissue sample expresses TIRC7 compared to a control cell. The immune cell is preferably a TIL.

The present invention is based on the finding that solid cancer patients surprisingly show different cellular expression of TIRC7, a major key regulator of proliferative pathways in immune cells. Cancer cells often use surrounding immune cells for their purposes to mask themselfes from the hosts immune system. The present invention now provides a new treatment approach by targeting tumor infiltrated immune cells via TIRC7 in order to overcome the immune escape of the tumor. According to the herein disclosed invention, TIRC7 can be used to stratify solid cancers into a responder and a non-responder group with respect to a TIRC7 modulatory treatment targeting immune cells. Therefore, the present invention for the first time provides a true theranostic approach (a combined diagnostic and therapeutic approach) for patients with solid cancers.

The methods of the invention may additionally in step (b) comprise: determining the expression of TIRC7 together with at least one additional immune cell factor selected from the group consisting of FoxP3 and CD20.

In context of the present invention it may be preferred that a TIRC7 modulatory treatment is a TIRC7 treatment that involves the activation of an TIRC7 dependent apoptotic signalling cascade. TIRC7 may induce apoptosis via signalling initiated by the binding of the TIRC7 ligand HLA DR alpha 2 in immune cells in the cancer tissue, for example Tregs, to enhance immune response against tumor and/or inhibit tumor cell growth via direct tumor killing. Therefore in preferred embodiments of the herein described invention the TIRC7 targeting comprises the modulation of the TIRC7-HLA-DR alpha 2 axis or signalling.

Correspondingly, the invention with respect to the term “TIRC7 modulator” preferably refers to a compound, which is selected from any compound that modulates TIRC7 expression, stability and/or function, and even more preferably a compound which modulates the signalling of the TIRC7 HLA-DR alpha 2 axis. A TIRC7 modulator may in some preferred embodiments of the invention be selected from that mimics HLA-DR alpha 2 mediated TIRC7 signalling. Such TIRC7 modulators may be selected from a nucleic acid, a protein, a small compound. Preferred inhibitors of TIRC7 of the invention include anti-TIRC7 antibodies, such as for example any of the antibodies disclosed in WO 99/11782, WO 03/054019 and WO 03/054018 (all incorporated herein in their entirety). A preferred antibody is Metiliximab (disclosed in WO 03/054019), or any chimerized, humanized or derivatized variants or fragments thereof, wherein the variant still comprises the CDR1 to CDR3 regions of the parent molecule. Alternatively the invention as TIRC7 modulator provides HLA-DR alpha 2 encoding nucleic acids or recombinant proteins, or functional variants or fragments thereof.

In some embodiments, the herein disclosed methods are ex vivo or in vitro methods, preferably wherein the methods are conducted completely ex vivo or in vitro.

The present invention provides a combined diagnostic and therapeutic approach for patients with solid tumors e.g deriving from colon, rectum, pancreas, head & neck, NSCLC, skin (Melanoma) or kidney. In a preferred embodiment the tumor or cancer is a solid tumor or cancer which has developed an immune escape via the hosts immune cells. Therefore, the cancer or tumor preferably is a solid tumor characterized in that the tumor tissue comprises tumor infiltrating lymphocytes (TIL), such as Tregs.

Accordingly, the present invention provides approaches for treating or preventing and/or diagnosing the growth and/or metastasis of solid cancers in a subject. As used herein, a “solid cancer” refers to one or more cells, which are growing or have grown in an uncontrolled manner to form cancer tissue. As used herein, the term “solid cancer” includes, but is not limited to “carcinomas”, “adenocarcinomas” and “sarcomas”. “Sarcomas” are cancers of the connective tissue, cartilage, bone, muscle, and so on. “Carcinomas” are cancers of epithelial (lining) cells. “Adenocarcinoma” refers to carcinoma derived from cells of glandular origin. The terms “cancer” and “tumor” are used interchangeably throughout the subject specification.

Solid cancers may arise in nearly any tissue of the body and the treatment of any solid cancer is contemplated by the present invention. Exemplary “solid cancers” which may be treated in accordance with the present invention include AIDS related cancer, acoustic neoma, adenocystic carcinoma, adrenocortical cancer, agnogenic myeloid metaplasia, alopecia, alveolar soft-part sarcoma, anal cancer, angiosarcoma, aplastic anaemia, astrocytoma, ataxia-telangiectasia, basal cell carcinoma (bcc), bladder cancer, bone cancers, bowel cancer, brain stem glioma, brain and CNS cancers, breast cancer, CNS cancers, carcinoid cancers, cervical cancer, childhood brain cancers, childhood cancer, childhood soft tissue sarcoma, chondrosarcoma, choriocarcinoma, colorectal cancers, cutaneous T-CeIl lymphoma, dermatofibrosarcoma-protuberans, desmoplastic small round cell cancer, ductal carcinoma, endocrine cancers, endometrial cancer, ependymoma, esophageal cancer, Ewing's sarcoma, extra hepatic bile duct cancer, eye cancer, eye: melanoma, retinoblastoma, fallopian tube cancer, fanconi anaemia, fibrosarcoma, gall bladder cancer, gastric cancer, gastrointestinal cancers, gastrointestinal carcinoid cancer, genitourinary cancers, germ cell cancers, gestational trophoblastic disease, glioma, gynecological cancers, hematological malignancies, head and neck cancer, hepatocellular cancer, hereditary breast cancer, histiocytosis, Hodgkin's disease, human papillomavirus, hydatidiform mole, hypercalcemia, hypopharynx cancer, intra-ocular melanoma, isle T-cell cancer, Kaposi's sarcoma, kidney cancer, Langerhan's cell histiocytosis, laryngeal cancer, leiomyosarcoma, li-fraumeni syndrome, lip cancer, liposarcoma, liver cancer, lung cancer, lymphedema, lymphoma, Hodgkin's lymphoma, non-Hodgkin's lymphoma, male breast cancer, malignant rhabdoid cancer of kidney, medulloblastoma, melanoma, merkel cell cancer, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, nijmegen breakage syndrome, non-melanoma skin cancer, non-small cell lung cancer (nscic), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral neuroectodermal cancers, pituitary cancer, polycythemia vera, prostate cancer, rare cancers and associated disorders, renal cell carcinoma, retinoblastoma, rhabdomyosarcoma, rothmund Thomson syndrome, salivary gland cancer, sarcoma, schwannoma, sezary syndrome, skin cancer, small cell lung cancer (scic), small intestine cancer, soft tissue sarcoma, spinal cord cancers, squamous cell carcinoma (sec), stomach cancer, synovial sarcoma, testicular cancer, thymus cancer, thyroid cancer, transitional cell cancer (bladder), transitional cell cancer (renal-pelvis−/−ureter), trophoblastic cancer, urethral cancer, urinary system cancer, uroplakins, uterine sarcoma, uterus cancer, vaginal Cancer, vulva cancer, Waldenstrom's macroglobulinemia and Wilms' Cancer.

The solid cancer which is treated using the methods of the present invention may be a primary lesion or may be the result of metastasis of a primary cancer. Furthermore, if the solid cancer is a metastasis of a primary cancer, the primary cancer may be either a primary solid cancer as described above or may be a dispersed primary cancer such as a leukemia or lymphoma.

The diagnostic approach of the present invention requires in step (b) a determining of the expression of TIRC7 in or on the tumor cell or in or on an immune cell, such as a TI1, in the tumor tissue. In some preferred embodiment of the invention the TIRC7 expression may be determined on the TIRC7 protein level, for example by using an anti-TIRC7 antibodies or mass spectroscopic methods, or at the mRNA level, for example using a PCR-based detection method or nucleic acid based hybridization technique. Any method known to the skilled person to determine the expression of a protein on a cell may be used in context of the present invention.

In preferred embodiments of the present invention, said control cell used in the methods is a cell not expressing TIRC7 protein (therefore is a negative control). Alternatively to a negative control, the method of the invention may comprise a comparison of the determined level of TIRC7 expression on the tumor cell with a reference value of a TIRC7 expression value that corresponds to the level of TIRC7 in a negative control.

A sample according to the present disclosure which comprises a tumor cell is preferably a tissue sample, wherein the tissue sample is obtained from a primary tumor or a tumor metastasis.

In some embodiments of the present disclosure the solid cancer patient is a re-lapsed or refractory cancer patient in which a previous therapy with a compound selected from the group consisting of an anti-CD20 antibody such as Rituximab; or check point inhibitors, any targeted agents and chemotherapies, has failed, or wherein the solid cancer is refractory and has become resistant to any of the aforementioned treatments.

Another aspect of the invention then pertains to a modulator of T cell immune response cDNA 7 (TIRC7) for use in the treatment of solid cancer in a patient. In this aspect, the above mentioned definitions equally apply. In particular, in some embodiments it is preferred that the solid cancer is a solid cancer disease in a late stage and/or a refractory cancer. A refractory cancer disease may be a disease that was previously treated with state of the art chemotherapeutic method either successfully or not, and which subsequently developed a resistance to chemotherapeutic agents selected from chemotherapeutic agents excluding TIRC7 modulators as defined and described in the present disclosure.

In context of the present disclosure, the treatment preferably comprises a preceding stratification of the patient suffering from solid cancer. Such a stratification is a stratification of the patient into one of patient groups (i) or (ii), wherein patient group (i) is a solid cancer patient group that will benefit from a T cell immune response cDNA 7 (TIRC7)-modulatory treatment, and patient group (ii) is solid cancer patient group that will not benefit from a TIRC7 modulatory treatment. The stratification is preferably a method as disclosed herein above.

Also provided are pharmaceutical compositions for use in the herein disclosed medical applications, comprising a TIRC7 modulator as described before together with a pharmaceutical acceptable carrier and or excipient. The term “pharmaceutical composition” refers to a preparation which is in such form as to permit the biological activity of an active ingredient contained therein to be effective, and which contains no additional components which are unacceptably toxic to a subject to which the composition would be administered. A pharmaceutical composition of the present invention can be administered by a variety of methods known in the art. As will be appreciated by the skilled artisan, the route and/or mode of administration will vary depending upon the desired results. To administer an antibody according to the invention by certain routes of administration, it may be necessary to coat the antibody with, or co-administer the antibody with, a material to prevent its inactivation. For example, the antibody may be administered to a subject in an appropriate carrier, for example, liposomes, or a diluent. Pharmaceutically acceptable diluents include saline and aqueous buffer solutions. A “pharmaceutically acceptable carrier” refers to an ingredient in a pharmaceutical formulation, other than an active ingredient, which is nontoxic to a subject. Pharmaceutically acceptable carriers includes any and all solvents, dispersion media, coatings, antibacterial and antifungal agents, isotonic and absorption delaying agents, and the like that are physiologically compatible. In one preferred embodiment, the carrier is suitable for intravenous, intramuscular, subcutaneous, parenteral, spinal or epidermal administration (e.g. by injection or infusion).

The pharmaceutical compositions according to the invention may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art. Actual dosage levels of the active ingredients in the pharmaceutical compositions of the present invention may be varied so as to obtain an amount of the active ingredient, which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The composition must be sterile and fluid to the extent that the composition is deliverable by syringe. In addition to water, in one embodiment the carrier is an isotonic buffered saline solution. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. An “immunoconjugate” is an antibody conjugated to one or more heterologous molecule(s), including but not limited to a cytotoxic agent.

The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Dosages will vary but a preferred dosage for intravenous administration of DNA is from approximately 10<6> to 10<12>copies of the DNA molecule. The compositions of the invention may be administered locally or systemically. Administration will generally be parenterally, e.g., intravenously; DNA may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like. Furthermore, the pharmaceutical composition of the invention may comprise further agents such as interleukins or interferons depending on the intended use of the pharmaceutical composition.

In another aspect, the invention pertains to a kit, comprising means for use in the combined diagnostic and therapeutic approach of the present invention. The kit preferably comprises (a) means for the determination of TIRC7 expression and (b) a TIRC7 modulator as a therapeutic. Optionally the kit may further comprise instructions for use and/or other agents that are helpful in the diagnosis or therapy of solid cancer.

Furthermore provided are methods for the treatment of a patient suffering from a solid cancer disorder, the method comprising a step of administering a therapeutically effective amount of an TIRC7 modulator as described herein before. Preferably, the TIRC7 modulator is a TIRC7 ligand, or derivative thereof, or an anti-TIRC7 antibody, or derivative or antigen binding fragment thereof, as described above.

The present invention will now be further described in the following examples with reference to the accompanying figures and sequences, nevertheless, without being limited thereto. For the purposes of the present invention, all references as cited herein are incorporated by reference in their entireties. In the Figures:

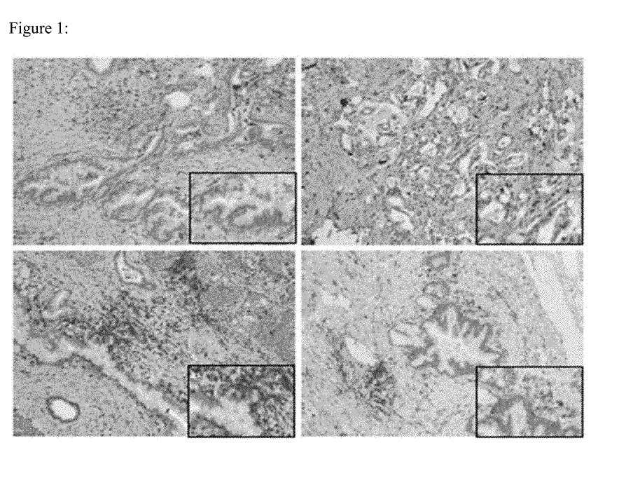

FIG. 1: TIRC7 expression is induced in human pancreatic carcinoma cells and TIL.

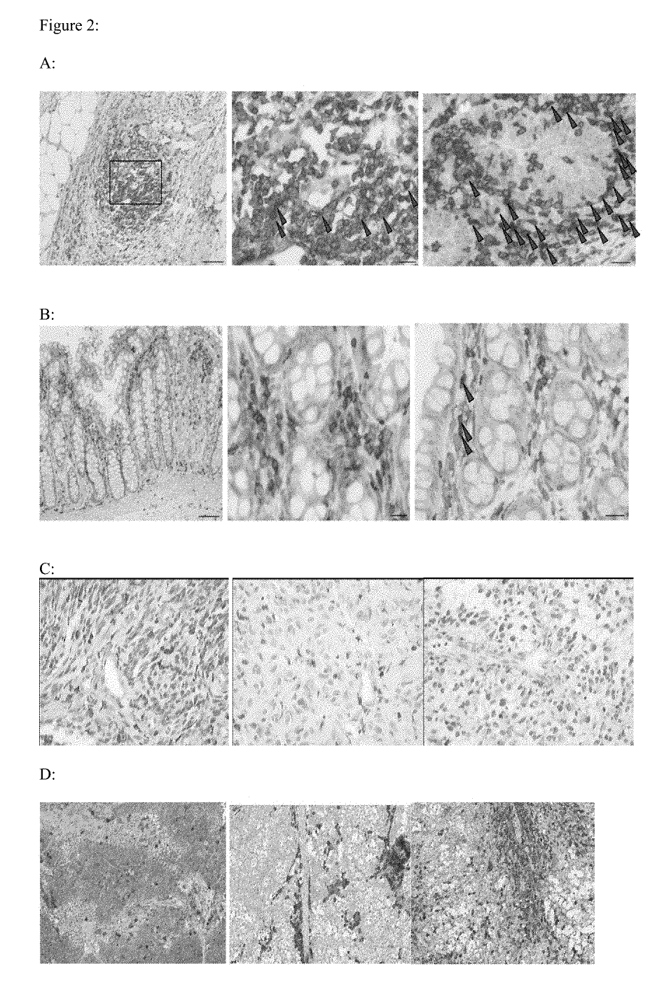

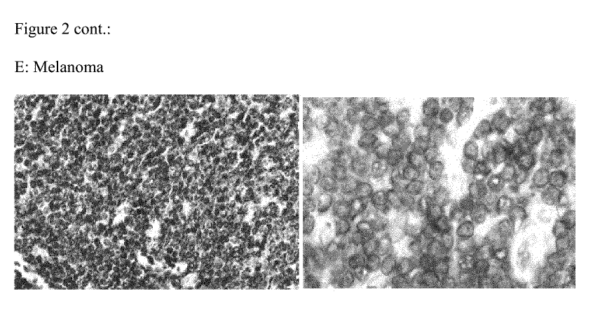

FIG. 2: TIRC7 expression is induced in TIL including Treg as shown in (A) colo-rectal carcinoma and (B) neuronal intestinal dysplasia (C) Glioblastoma (left and middle panel) and anaplastic oligodendroglioma (right panel) (D) Merkel cell carcinoma (E) melanoma

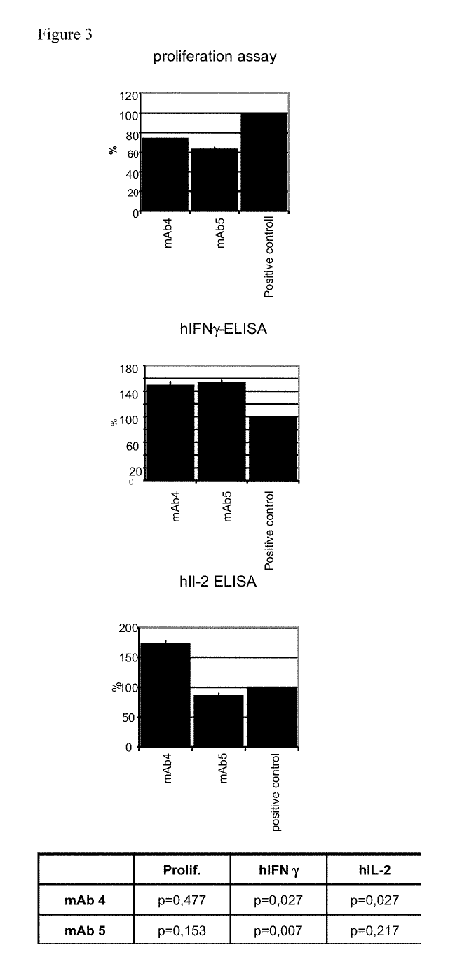

FIG. 3: Anti-TIRC7 mAb stimulates proliferation and interferon gamma expression of human lymphocytes

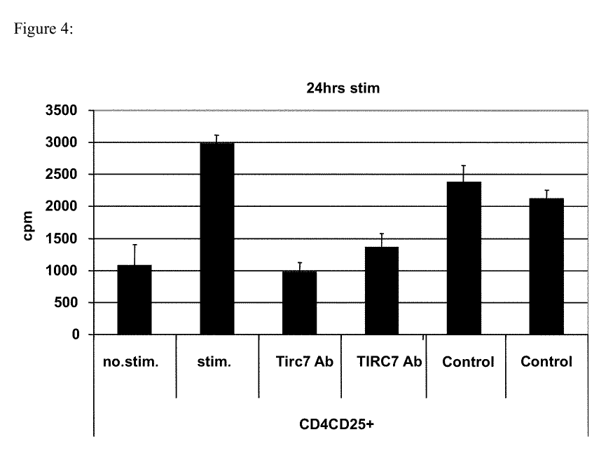

FIG. 4: Anti-TIRC7 mAbs inhibit proliferation of T regulatory cells (Treg)

### EXAMPLES

FIG. 1 shows that TIRC7 expression is induced in human pancreatic carcinoma cells and TIL infiltrating the tumor tissue. Then anti-Tirc7 antibody (metiliximab) was used to stain tumors derived from various patients with cancer according to standard immune histo-staining procedures of formalin embedded tissues (Bulwin et al, Plos One 2007). Results are shown in FIG. 2.

To examine the stimulatory effect of the anti-TIRC7 mAb on lymphocytes to enhance the immune response against tumor antigens were incubated in the presence and absence of anti-Tirc7 antibody or control antibody and subjected to proliferation assays. Results are shown in FIG. 3.

To examine the inhibitory effect of the anti-TIRC7 mAb on Treg spleen Tregs were incubated in the presence and absence of anti-Tirc7 antibody or control antibody and subjected to proliferation assays. Animal: Balb/c (Age: 12 weeks). CD4CD25 cell were isolated by using microbeads isolation kit from naive splenocytes, and stimulated with PHA and IL2 for 24 h. Cells were cultured under 5% CO2, 37 C for 24 hrs with either chimeric anti-TIRC7mAb or negative control IgG at different concentrations. Proliferation was assessed by 3H-thymidine methods. The results are provided in FIG. 4.

1. An ex vivo method for stratifying a solid cancer patient into one of patient groups (i) or (ii), wherein patient group (i) is a solid cancer patient group that will benefit from a T cell immune response cDNA 7 (TIRC7)-modulatory treatment, and patient group (ii) is a solid cancer patient group that will not benefit from a TIRC7 modulatory treatment, the method comprising the ex vivo method steps of
(a) Providing a sample of tumor tissue of the solid cancer of the patient, wherein the tumor tissue comprises a tumor cell and/or a tumor infiltrating immune cell,
(b) Determining the expression of TIRC7 in or on the tumor cell and/or infiltrating immune cell, and
(C) Depending on the resultant of step (b), stratifying the patient into group (i) in the event the tumor cell in the tumor tissue and/or the immune cell in the tumor tissue expresses TIRC7 compared to a control cell, or stratifying the patient into group (ii) in the event the tumor cell in the tumor tissue and/or the immune cell in the tumor tissue does not express TIRC7 compared to a control cell.

2. An ex vivo method for diagnosing a cancer patient to have a solid cancer disease which will respond to a TIRC7 modulatory treatment, the method comprising the ex vivo method steps of
(a) Providing a sample comprising a tumor tissue from said tumor in the cancer patient,
(b) Determining the expression of TIRC7 in a tumor cell in the tumor tissue or in an immune cell, such as a TIL, in the tumor tissue,
(c) Depending on the resultant of step (b), diagnosing the patient to have a solid cancer disease which will respond to a TIRC7 modulatory treatment in the event the tumor cell in the tumor tissue and/or the immune cell in the tumor tissue expresses TIRC7 compared to a control cell.

3. The method according to claim 1 or 2, wherein step (b) comprises determining the expression of TIRC7 and at least one additional immune cell factor such as FoxP3.

4. The method according to any of claims 1 to 3, wherein TIRC7 modulatory treatment is an TIRC7 inhibitory treatment and wherein said TIRC7 modulator is a TIRC7 inhibitor.

5. The method according to any of claims 1 to 4, which is performed completely ex vivo, preferably in vitro.

6. The method according to any of claims 1 to 5, wherein the TIRC7 modulatory treatment is a treatment comprising the administration of a TIRC7 modulator, such as an antibody binding to, and modulating, TIRC7, preferably wherein the antibody is metiliximab, or antibody derivatives of this molecule.

7. The method according to any of claims 1 to 6, wherein in step (b) the expression of TIRC7 in or on the cancer tumor cell and/or immune cell is determined on the protein level, for example by using an anti-TIRC7 antibody, or at the mRNA level, for example by a PCR-based detection method or hybridization technique.

8. The method according to any of claims 1 to 8, wherein said control cell is a cell not expressing TIRC7 protein (negative control).

9. The method according to any of claims 1 to 8, wherein the cancer patient is a relapsed or refractory cancer patient in which a previous therapy with a compound selected from the group consisting of an anti-CD20 antibody such as Rituximab, fludarabine and chlorodeoxiadenosine, has failed.

10. A modulator of T cell immune response cDNA 7 (TIRC7) for use in the treatment of lymphoma solid cancer in a patient.

11. The modulator of TIRC7 for use according to claim 10, wherein the solid cancer is a TIRC7 expressing solid cancer, and/or is a solid cancer characterized by the presence of tumor infiltrating lymphocytes in the tumor tissue.

12. The modulator of TIRC7 for use according to any of claims 10 to 11, wherein the treatment comprises a preceding stratification of the patient suffering from the solid cancer disease.

13. The modulator of TIRC7 for use according to claim 12, wherein the stratification is a stratification of the patient into one of patient groups (i) or (ii), wherein patient group (i) is a solid cancer patient group that will benefit from a T cell immune response cDNA 7 (TIRC7)-modulatory treatment, and patient group (ii) is solid cancer patient group that will not benefit from a TIRC7 modulatory treatment, according to method of any of claims 1 to 9.

14. The modulator of TIRC7 for use according to any of claims 10 to 13, wherein the patient is a relapsed or refractory solid cancer patient in which a previous therapy with a compound selected from the group consisting of anti-CD20 antibody such as Rituximab, fludarabine and chlorodeoxiadenosine, has failed.

15. The modulator of TIRC7 for use according to any of claims 10 to 14, wherein the solid cancer is a cancer stemming from any solid organ tissue.

16. A method for treating a patient suffering from solid cancer, the method comprising the steps of
(a) Determining the expression of TIRC7 in or on a tumor cell and/or immune cell comprised in a tumor tissue sample from the patient compared to a negative control,
(b) If the resultant of step (a) is that TIRC7 is expressed in or on the tumor cell and/or immune cell, administering to the patient a therapeutically effective dose of a TIRC7 modulator.

17. The method according to claim 16, wherein step (a) comprises a method according to any of claims 1 to 9.

18. The method according to claim 16 or 17, wherein the modulator of TIRC7 is an antibody binding to, and inhibiting, the extracellular domain of TIRC7, preferably wherein the antibody, or antibody derivatives of these molecules, such as chimerized, humanized, or otherwise optimized antibody molecules.

19. The method according to any of claims 16 to 18, wherein the patient is a relapsed or refractory solid cancer patient in which a previous therapy with a compound selected from the group consisting of anti-CD20 antibody such as anti-PD1. PD1L, CTLA4 or other targeted therapies, has failed.

20. The method according to any of claims 16 to 19, wherein the therapeutically effective dose of a TIRC7 modulator induces an increased caspase 3 dependent apoptosis in tumor cells in said patient.
